(19) United States
(12) Patent Application Publication
(10) Pub. No.: US 2016/0278824 A1
(43) Pub. Date: Sep. 29, 2016

(54) METHOD FOR TREATING FRACTURES OF A BONE

(71) Applicant: MEDARTIS HOLDING AG, Basel (CH)

(72) Inventor: Fatih TOY, Muttenz (CH)

(21) Appl. No.: 14/668,244

(22) Filed: Mar. 25, 2015

(51) Int. Cl. A61B 17/80; A61B 34/10

(52) U.S. Cl. CPC A61B 17/80; A61B 34/10; A61B 2017/564

(57) ABSTRACT

A method for treating fractures of a bone by plate osteosynthesis comprises the steps of:
determining at least one parameter representing the bone size,
classifying the bone based on the at least one parameter representing the bone size to obtain a classification,
assigning a screw configuration for fixing a bone plate to the bone based on the classification,
determining at least one additional parameter representing the bone size,
confirming the suitability of the screw configuration assignment based on the at least one additional parameter representing the bone size,
using the screw configuration for fixing the bone plate to the bone, if the suitability of the screw configuration assignment is confirmed based on the at least one additional parameter representing the bone size.

### BACKGROUND OF THE INVENTION

The present invention relates to a method for treating fractures of a bone by plate osteosynthesis according to the preamble of claim 1.

A well established and highly successful procedure employed in the treatment of bone fractures involves the use of bone stabilising implants for reliable stabilisation of a broken bone in its normal position. Such implants, e.g. bone plates, are usually made from metal, in particular of titanium or surgical steel. During treatment, they are fixed to the bone parts by means of threaded screws, which are driven into the bone tissue. By providing such an internal fixation to a patient, four major issues are addressed: Anatomic reduction of the fracture, stable fixation of the bone, preservation of blood supply and early, active mobilisation to previous function.

A typical surgical procedure for the fixation of a bone fracture, in particular of a long bone, such as the femur, tibia, humerus or radius, involves the formation of a surgical approach through a skin and tissue incision. After reducing the fracture, a bone plate is placed on the fractured bone. This plate is then secured with fixation elements, such as screws and/or K-wires. The bone plate stabilizes the fracture and keeps the bone in the correct position so as to allow the fracture to heal. Typically, bone plates have a bone contacting side and a side facing away from the bone with a plurality of holes or apertures extending between the two surfaces. The screws may be used either bi- or monocortically. If a chosen bicortical screw is too long, it may create trauma to soft tissue on the opposing cortex. If a chosen monocortical screw is too short, the plate-screw-construct may not withstand post-operative loads. It is therefore very important to choose the right screw length for the case at hand.

In order to reduce operative trauma to the patient and allow for rapid remobilisation, a minimally invasive procedure is desirable. The technical documentation “LISS DF” by Synthes, Inc. of Switzerland (published May 2008) describes a less invasive stabilisation system for distal femur fractures. The document covers different plate osteosynthetic procedures, including, among others, the insertion of self-drilling monocortical screws or the insertion of self-tapping bicortical screws. In this context, it is described that the employment of monocortical screws allows for pre-operative screw lengths selection using an anterior-posterior radiograph. To this end, a pre-operative X-ray of the fractured bone is performed with a calibrator placed medially or laterally at the height of a condyle. The size of the calibrator and of the condyle is then measured in the X-ray image. Based on these measurements, a screw configuration can be assigned to the fractured bone, defining an individual screw length for each hole of the bone plate.

This method is not amenable in cases where bicortical screws are used, due to the risk of perforation of the opposing cortex. The insertion of bicortical screws therefore requires pre-drilling of all screw holes, at least in the diaphyseal region. The drilled depth can then be measured for each screw hole to assign the required screw length. This procedure is very time consuming. It prolongates the duration of surgery, and is therefore not only a burden to the patient, but also cost-intensive.

It is therefore a problem underlying the present invention, to overcome the aforementioned drawbacks in the prior art. In particular, it is a problem underlying the present invention to provide a method for the treatment of bone fractures by plate osteosynthesis, in which a screw configuration is assigned to a fractured bone with increased efficiency and safety. The method should be technically simple and cost efficient. Moreover, it should reduce the surgical burden to the patient.

These problems are solved by a method for treating fractures of a bone according to claim 1.

### SUMMARY OF THE INVENTION

Various aspects of the present invention are achieved by a method for treating fractures of a bone by plate osteosynthesis, the bone having a bone size, the method comprising the steps of:

Determining at least one parameter representing the bone size.

Classifying the bone based on the at least one parameter representing the bone size to obtain a classification.

Assigning a screw configuration for fixing a bone plate to the bone based on the classification.

Determining at least one additional parameter representing the bone size.

Confirming the suitability of the screw configuration assignment based on the at least one additional parameter representing the bone size.

Using the screw configuration for fixing the bone plate to the bone, if the suitability of the screw configuration assignment is confirmed based on the at least one additional parameter representing said bone size.

By using this technique, the risk of perforation of the opposing cortex is substantially eliminated and pre-drilling of all screw holes becomes obsolete. Although, for the sake of patient-safety, monocortical screws are preferably used in combination with the present invention, the method can even be employed when bicortical screws are used for fixation.

Consequently, a method for treating fractures of a bone by plate osteosynthesis is provided, which is significantly more efficient, cost-effective, and also safer. It reduces surgical burden and trauma to the patient and can allow for faster healing of a fracture. Furthermore, the risk of infection is reduced, as the amount of equipment, which comes in contact with the fractured bone, is decreased.

The step of determining the at least one additional parameter representing the hone size can comprise determining the distance between a point of entry and a point of exit of an axis crossing the bone in a direction being non-parallel to the longitudinal axis of said bone.

By way of example, a measuring bow can be used for determining the distance between the point of entry and the point of exit of the axis crossing said bone in a transverse direction. The use of a measuring bow constitutes a particularly simple and reliable measurement method.

A typical classification-scheme for classifying the hone based on the at least one parameter representing the bone size can be S (small), M (medium) or L (large). However it is also possible to employ a finer scheme, such as XXS (extra-extra-small), XS (extra-small), S (small), M (medium), L (large), XL (extra-large) or XXL (extra-extra-large).

On the other hand, the step of determining said additional parameter representing said bone size can also comprise:

Drilling a reference bore into the bone.

Determining at least one spatial dimension of the reference bore.

This allows for the determination of the additional parameter representing the bone size in a particularly reliable manner, as the measurement is performed exactly at the place where a screw is eventually placed. However, only one hole has to be drilled into the bone.

Irrespective of whether or not a reference bore is drilled into the bone, a drilling block can be used in the step of determining the additional parameter representing the bone size. The drilling block can be positioned on a pre-selected bone plate or directly on the bone. As one-dimensional measurements of bone-dimensions are usually dependent on their direction, a precise definition of the measurement-path by use of a drilling block generally allows for more reliable measurements.

Preferably, the method is used with a screw configuration comprising self-drilling and self-tapping screws. In this case, no pilot holes are required. Since the suitability of the pre-selected screw configuration is confirmed, the risk of perforation of the opposing cortex is substantially eliminated.

If a reference bore is employed, it is preferably drilled fully through the bone. Furthermore, the step of determining at least one spatial dimension of the reference bore can comprise determining the length of the reference bore. This can be achieved in several alternative ways. One option is the use of a separate measuring device for determining the length of the reference bore. On the other hand, it is also possible to use a specialized drilling device for simultaneously drilling said reference bore and determining its length. This can be achieved, for instance, with a depth-scale on the drill. This option is particularly attractive, as it allows for drilling the reference bore and measuring its length in one single step.

The method can comprise the further step of assigning a bone plate based on the classification. By this means, the information that is gained by determining the at least one parameter representing the bone size is not only used for assigning the screw configuration, but also for selecting the size of the bone plate.

Furthermore, an imaging technique can be used for determining the at least one parameter representing the bone size. Imaging methods are widely used in the clinical environment and allow for the determination of the spatial dimensions of a bone in high precision, as no soft-tissue is interfering with the measurement. Said imaging techniques can be selected from the group consisting of X-ray, CT and MRI. This kind of equipment is available at every principal medical facility, e.g. a hospital. These techniques are used as standard diagnostic methods in connection with all kinds of bone fractures. Consequently, the data obtained by making a diagnosis can be used at a later stage for the determination of the bone size. Furthermore, the medical staff is usually trained and experienced in using imaging techniques. In a preferred embodiment of the present invention, a template, for instance an X-ray template, is used for determining the at least one parameter representing the bone size. In this context, the template cannot only be used for determining the bone size, but also to perform the classification of the bone based on this parameter in one step. This makes the procedure even more effective and cost efficient.

The fractured bone can be a long bone, in particular a femur, a tibia, a humerus or a radius. Furthermore, a method according to the present invention is amenable for the treatment of periarticular fractures.

By way of example, the long bone can be a radius. The step of using the screw configuration for fixing the bone plate can involve mounting the bone plate on a surface of the radius, wherein said surface is selected from the group consisting of volar, lateral, medial and dorsal. Moreover, the step of using the screw configuration for fixing the bone plate can involve mounting the bone plate on one of the distal or proximal ends of the radius. This underlines the flexibility of the method according to the present invention for the treatment of fractures.

The step of assigning a screw configuration for fixing a bone plate can involve addressing a particular screw length to each plate hole intended to receive a screw. In this manner, a stable fixation of the bone plate can be achieved with a minimal risk of perforation of the opposing cortex.

The step of determining the at least one parameter representing the bone size can constitute a determination in a first direction, wherein the step of determining the at least one additional parameter representing said bone size constitutes a determination in a second direction. This way, a cross-check over two dimensions is performed to minimise the risk of perforation of the opposing cortex. Said first and second directions can be non-parallel to each other. Preferably said first and second directions can be orthogonal to each other.

The method according to the present invention can also comprise the further step of subjecting the bone to an imaging technique after treatment for detecting any screw perforation. Such a screw-perforation can occur, for instance, at the dorsal or volar side of a bone, if the fractured bone has rather unusual dimensions. This post-operative control allows assessing the success of the treatment. Furthermore, any damage caused by the screws used to fix the bone plate can be detected and, if necessary, repaired.

### BRIEF DESCRIPTION OF THE DRAWINGS

FIG. 1 shows a flow chart of the method according to the present invention.

FIG. 2 shows yet another flow chart of the method according to the present invention with the employment of a reference bore.

FIG. 3 shows the determination of a parameter representing the bone size.

FIG. 4 shows a scheme for classifying a fractured bone based on the parameter representing the bone size according to FIG. 3.

FIG. 5 shows a cross-sectional view of a bone and a bone plate according to the present invention.

FIG. 6 shows a cross-sectional view of a bone with a reference bore and a bone plate according to the present invention.

FIG. 7 shows a top view of a bone and a bone plate according to FIGS. 5 and 6.

FIG. 8 shows a cross-sectional view of a bone plate and a drilling block according to an embodiment of the present invention.

FIG. 9 shows a cross-sectional view of a bone with a reference bore, a bone plate and a drilling block according to an embodiment of the present invention.

FIG. 10 shows a top view of a bone, a bone plate and a drilling block according to FIGS. 8 and 9.

FIG. 11 shows an example of a screw configuration.

FIG. 12 shows a further example of a screw configuration.

FIG. 13 shows yet another example of a screw configuration.

### DETAILED DESCRIPTION OF THE DRAWINGS

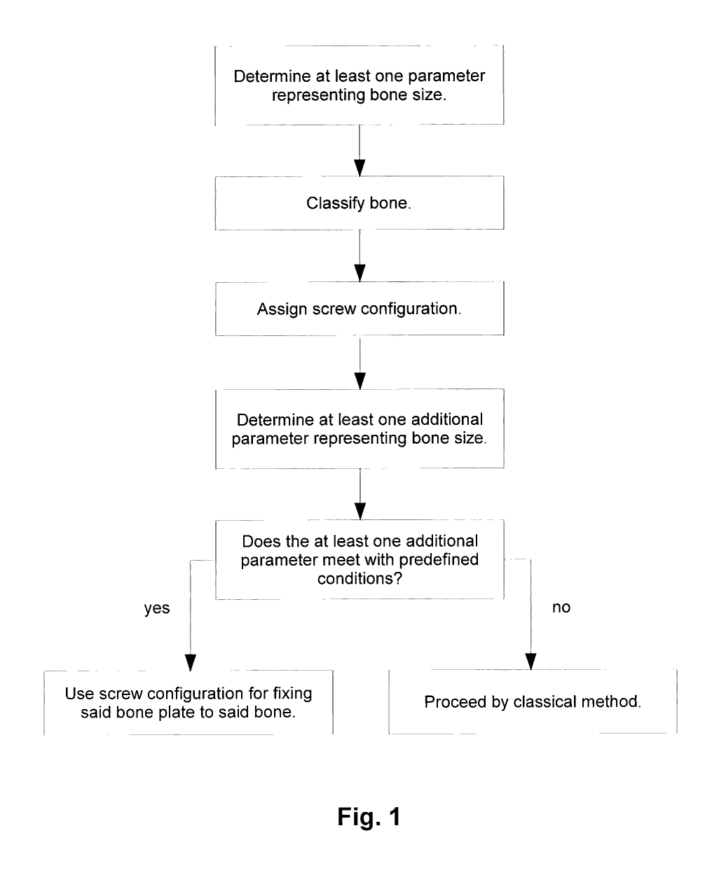

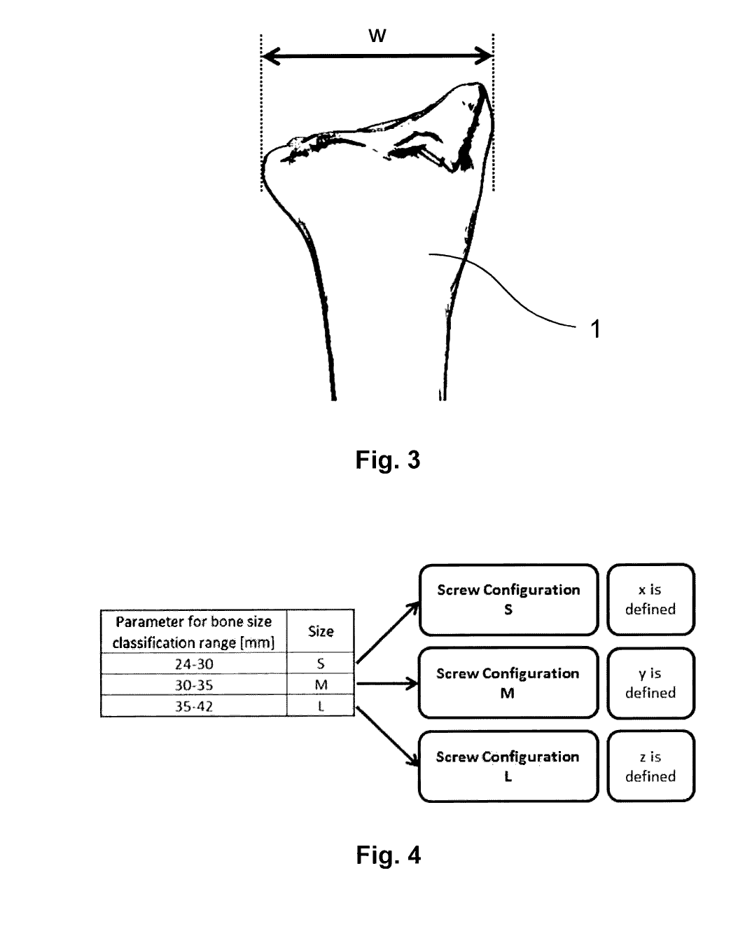

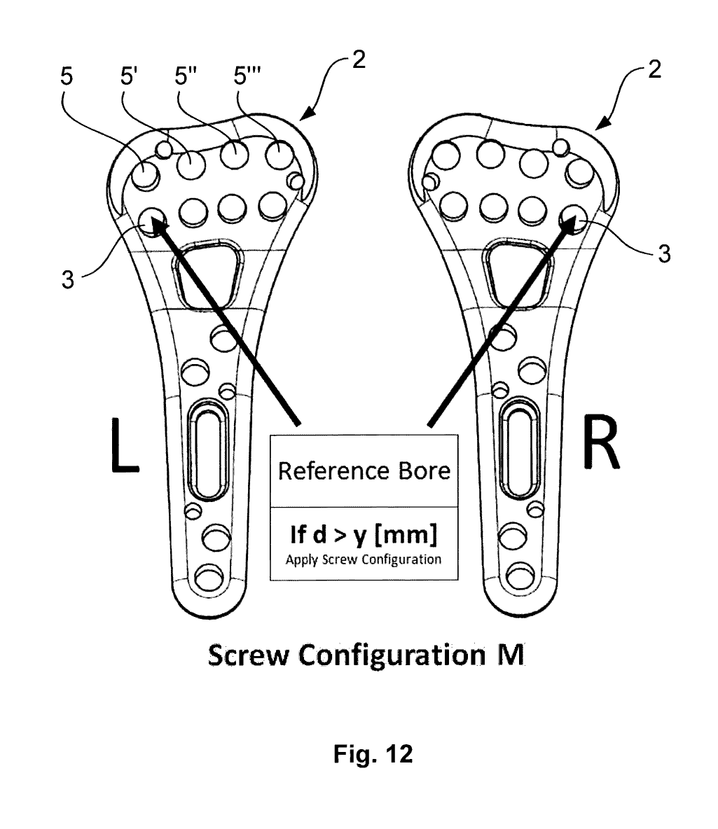

Referring to FIG. 1, there is shown a flow chart of a method according to the present invention. According to this flow chart, the first step of this method for treating fractures of a bone 1 by plate osteosynthesis involves the determination of at least one parameter representing the bone size. By way of example, as detailed in FIG. 3, this parameter can be the width w of the epiphysis distalis radii (distal width). In a next step, the fractured bone is classified based on this parameter to obtain a classification (e.g. medium, cf. FIG. 4). Based on this classification, a configuration of screws for fixing a bone plate 2 is assigned. For the case of a medium-sized radius, for instance, a screw configuration according to FIG. 12 can be assigned. Next, at least one additional parameter representing the bone size is determined. This can be achieved, for instance, by measuring the diameter or the thickness, respectively, of the bone at a pre-defined place and direction with a measuring bow. In the following step, the suitability of the screw configuration assignment is confirmed according to predefined conditions. If the screw configuration is suitable for fixing the bone plate, it is used accordingly. In case the configuration turns out not to be suitable for fixing the bone plate, the surgeon has to proceed with the classical method. This involves drilling a separate pilot hole for each individual screw and measuring its depth in order to determine the required length of the screw.

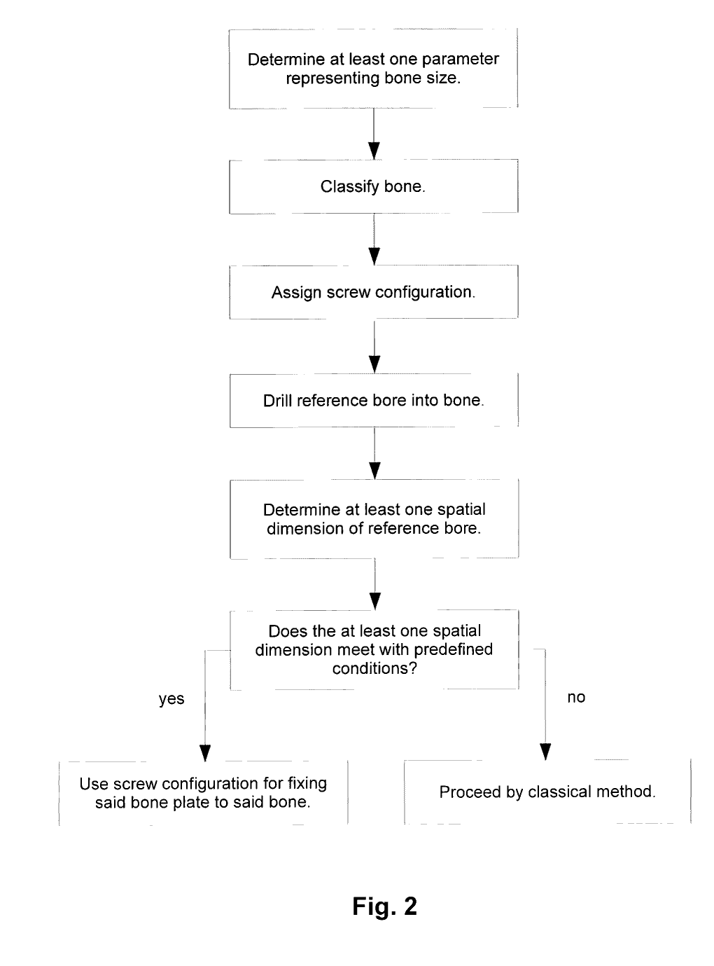

Referring to FIG. 2, a flow chart of a method according to the present invention is shown, wherein a reference bore is employed. The reference bore 4 is drilled into the bone and at least one spatial dimension d of the reference bore 4 is determined. The reference bore 4 can be drilled through a special hole 3 in the bone plate, but other plate holes (e.g. 5, 5′, 5″, 5′″) can also be suitable. However, when another plate hole is used, different confirmation criteria might apply. In order to ensure that the reference bore is drilled in a proper direction, a drilling block 6 can be used. The steps of drilling the reference bore 4 and determining the at least one spatial dimension d of the reference bore 4 can be carried out in sequence, as shown on the present flow chart, for instance with a standard bone drill and a depth gauge. But they can also be carried out as one single step, for instance with a drilling device that comprises a depth scale. In case of the screw configuration according to FIG. 12, the depth of the reference bore has to be deeper than y. Hence, if d>y the screw configuration can be used accordingly. In case d<y, the surgeon has to proceed with the classical method, as described above.

Referring to FIG. 3, determination of a parameter representing the bone size is shown based on the width w of the epiphysis distalis radii (distal width). The arrow and the dotted lines indicate the exact position, at which the distal width w is measured. The measurement can be performed conveniently by computer-assisted processing on an anterior-posterior radiograph.

Referring to FIG. 4, classification of the bone 1 based on the parameter w in FIG. 3 and assignment of a screw configuration is detailed. It can be seen that the classification scheme defines three dimensional ranges in which the bone is classified, as small, medium or large. The classification of the bone 1 also provides the conditions that have to be fulfilled by the reference bore in order to allow for the use of a given screw configuration.

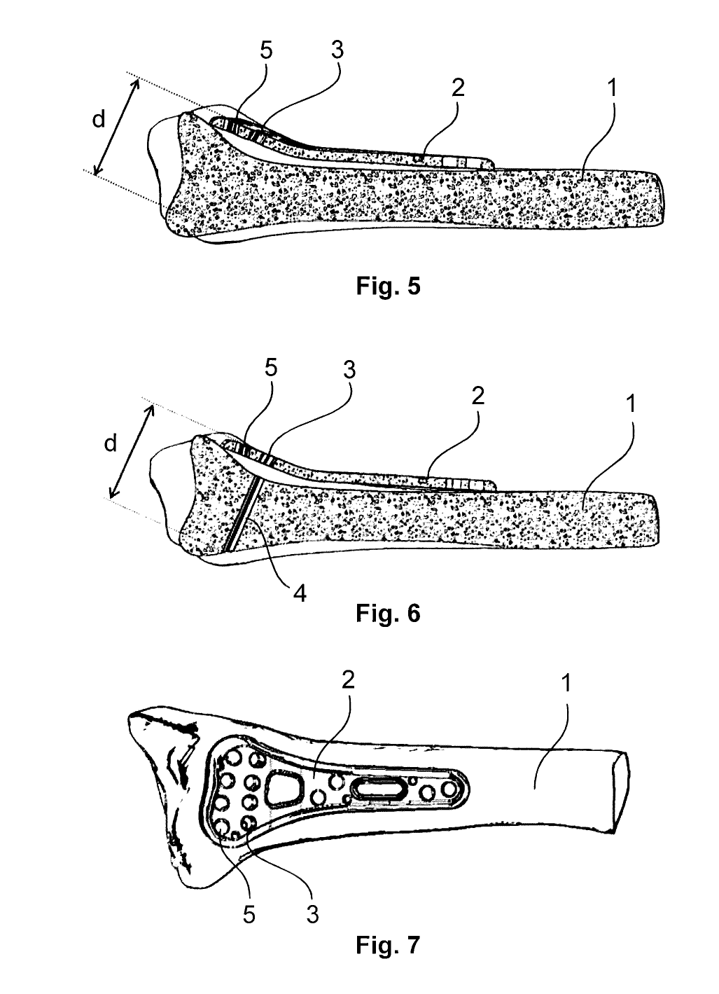

Referring to FIGS. 5 to 7, a cross-sectional view and a top view of a bone 1 and a bone plate 2 according to the present invention are shown. Indication of a fracture of bone 1 has been omitted for clarity. However, FIGS. 5 to 7 have to be understood such that bone 1 is fractured at a minimum of one site. In the present case, reference bore 4 is drilled through a special hole 3 in bone plate 2 for measuring the spatial dimension d (FIG. 6). Alternatively, a measuring bow might be positioned in the special hole 3 in bone plate 2 in order to determine the spatial dimension d without drilling (FIG. 5).

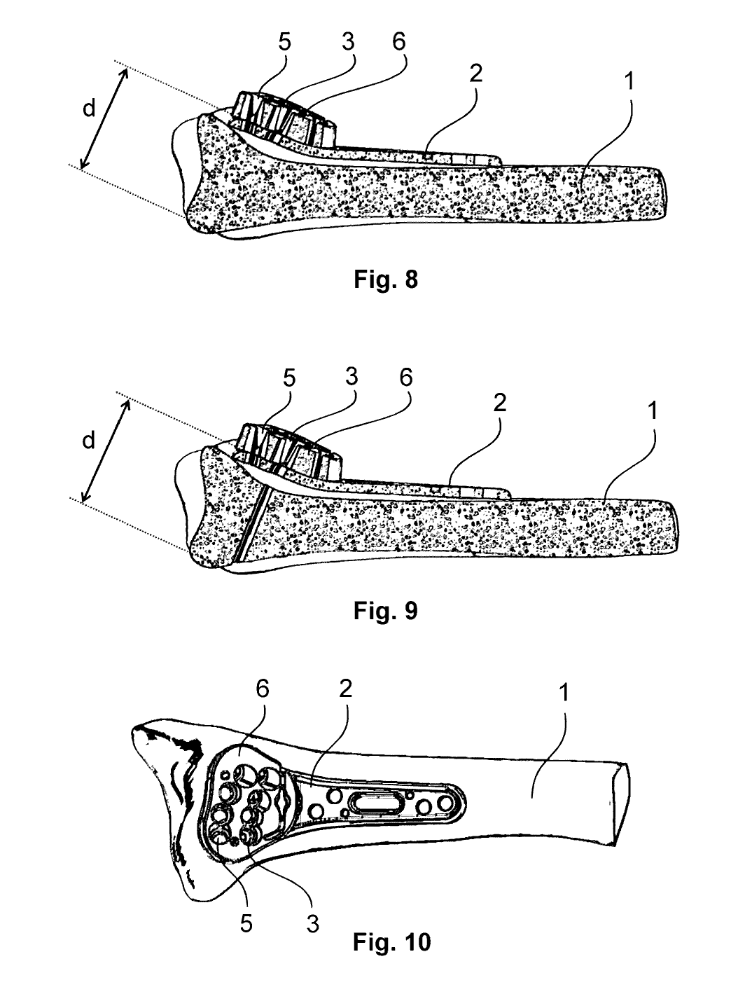

Referring to FIGS. 8 to 10, a drilling block 6 is additionally shown. The drilling block 6 can be used to ensure proper alignment of a reference bore (FIG. 9) or a measuring bow for determining at least one additional parameter representing the bone size (FIG. 8). For the sake of clarity, no measuring or drilling devices are shown in the figures. A drilling block 6 can also be beneficial to achieve a proper orientation of the screws holding the bone plate.

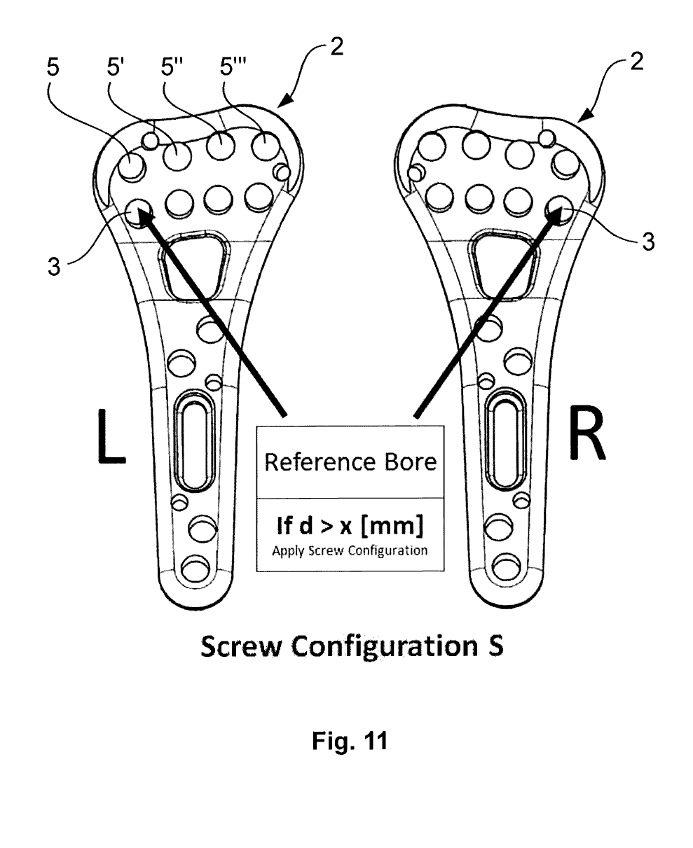

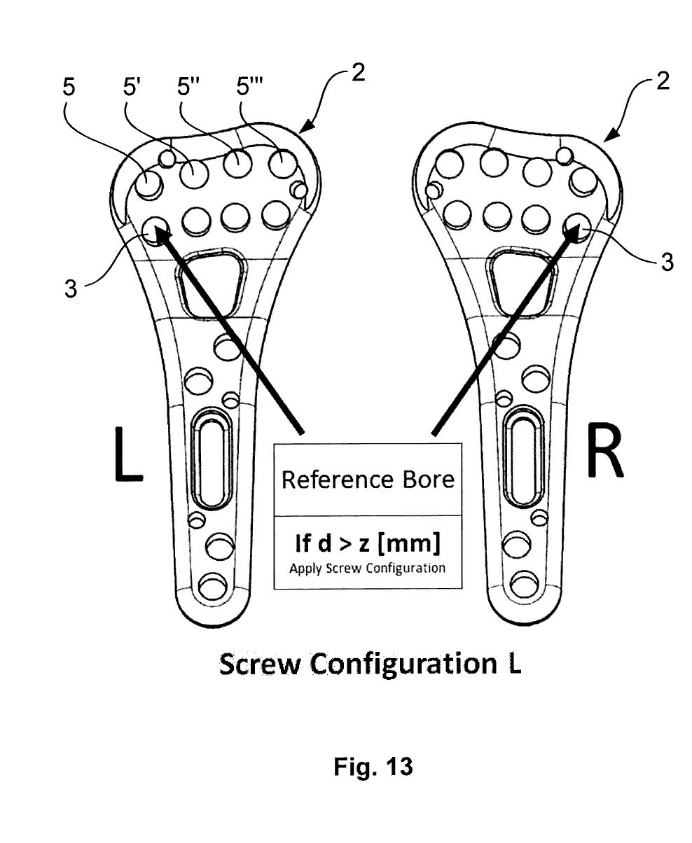

Referring to FIGS. 11 to 13, three possible screw configurations according to the classification scheme in FIG. 4 are shown. The screw configuration in FIG. 11 refers to a small sized bone. The pair of bone plates 2 shown refers to the left and right side. The plates 2 are suitable for fixation at the volar surface of a distal radius 1. For each plate hole (5, 5′, 5″, 5′″, etc.), a particular screw size and type is defined in the configuration. Furthermore, there is a hole 3 specified to drill the reference bore or for measuring the additional parameter representing the bone size by means of a measuring bow. Moreover, there is a condition for the use of the screw configuration provided, namely that the spatial dimension d of the reference bore has to be greater than x (d>x). FIGS. 12 and 13 show analogous screw configurations for radii that are classified as medium or large according to the scheme in FIG. 4. It can be seen that the conditions for using these screw configurations are defined as d>y and d>z.

1. A method for treating fractures of a bone by plate osteosynthesis, the bone having a bone size, comprising the steps of:
determining at least one parameter representing said bone size
classifying said bone based on said at least one parameter representing said bone size to obtain a classification
assigning a screw configuration for fixing a bone plate to said bone based on said classification
determining at least one additional parameter representing said bone size
confirming the suitability of said screw configuration assignment based on said at least one additional parameter representing said bone size
using said screw configuration for fixing said bone plate to said bone, if the suitability of said screw configuration assignment is confirmed based on said at least one additional parameter representing said bone size.

2. The method of claim 1, wherein the step of determining said at least one additional parameter representing said bone size comprises determining the distance between a point of entry and a point of exit of an axis crossing said bone in a direction being non-parallel to the longitudinal axis of said bone.

3. The method of claim 2, wherein a measuring bow is used for determining said distance between said point of entry and said point of exit of said axis crossing said bone in a transverse direction.

4. The method of claim 1, wherein the step of determining said additional parameter representing said bone size comprises:
drilling a reference bore into said bone
determining at least one spatial dimension of said refer ce bore

5. The method of claim 1, wherein a drilling block is used in the step of determining said additional parameter representing said bone size.

6. The method of claim 4, wherein said reference bore is drilled fully through said bone.

7. The method of claim 6, wherein the step of determining at least one spatial dimension of said reference bore comprises determining the length of said reference bore.

8. The method of claim 7, wherein a separate measuring device is used for determining said length of said reference bore.

9. The method of claim 7, wherein a drilling device is used for simultaneously drilling said reference bore and determining its length.

10. The method of claim 1, comprising the further step of assigning a bone plate based on said classification.

11. The method of claim 1, wherein an imaging technique is used for determining said at least one parameter representing said bone size.

12. The method of claim 11, wherein said imaging technique is selected from the group consisting of X-ray, CT and MRI.

13. The method of claim 12, wherein a template is used for determining said at least one parameter representing said bone size.

14. The method of claim 1, wherein said bone is a long bone.

15. The method of claim 14, wherein said long bone is a radius.

16. The method of claim 15, wherein the step of using said screw configuration for fixing said bone plate involves mounting said bone plate on a surface of said radius, wherein said surface is selected from the group consisting of volar, lateral, medial and dorsal.

17. The method of claim 15, wherein the step of using said screw configuration for fixing said bone plate involves mounting said bone plate at one of the distal or proximal end of said radius.

18. The method of claim 1, wherein the step of assigning said screw configuration for fixing said bone plate involves assigning a particular screw length to each plate hole intended to receive a screw.

19. The method of claim 1, wherein the step of determining said at least one parameter representing said bone size constitutes a determination in a first direction and wherein the step of determining said at least one additional parameter representing said bone size constitutes a determination in a second direction.

20. The method of claim 19, wherein said first and said second direction are non-parallel to each other.

21. The method of claim 1, comprising the further step of subjecting said bone to an imaging technique after treatment for detecting any screw perforation.

22. The method of claim 16, wherein the step of using said screw configuration for fixing said bone plate involves mounting said bone plate at one of the distal or proximal end of said radius.
